(19) United States
(12) Patent Application Publication
(10) Pub. No.: US 2025/0288739 A1
(43) Pub. Date: Sep. 18, 2025

(54) Syringe Pressure Sensor Assembly

(71) Applicants: Becton, Dickinson and Company, Franklin Lakes, NJ (US); Ferrosan Medical Devices SP.ZO.O., Szczecin (PL)

(72) Inventors: Kevin M. Ryan, Whitehouse Station, NJ (US); Angela Marie Huenerfauth, Morris Plains, NJ (US); Paavana Sainath, Fairfield, NJ (US); Frederik Sonnenborg, Jyllinge (DK); Søren Falkesgaard Ørts, Virum (DK); Søren Christensen, Allerød (DK)

(21) Appl. No.: 18/861,232

(22) PCT Filed: May 4, 2023
(86) PCT No.: PCT/US2023/020935

§ 371 Date: Oct. 29, 2024

Related U.S. Application Data
(60) Provisional application No. 63/338,662, filed on May 5, 2022.

(51) Int. Cl. A61M 5/168; A61M 5/142; A61M 5/145; A61M 5/315

(52) U.S. Cl. CPC A61M 5/16859; A61M 5/14216; A61M 5/14546; A61M 5/16886; A61M 5/31505; A61M 2205/332

(57) ABSTRACT

A syringe pressure sensor assembly may include a syringe barrel, a plunger rod including an outer support structure and an inner force translation rod, and a stopper secured to the plunger rod. The stopper may include a flexible membrane including proximal face and a distal face opposite the proximal face and a sidewall surrounding the flexible membrane. The outer support structure may be secured to the sidewall of the stopper, and the inner force translation rod may contact the proximal face of the flexible membrane. The flexible membrane may move proximally relative to the sidewall in response to a proximally directed force applied to the distal face of the flexible membrane, and the inner force translation rod may transfer, in response to the proximal movement of the flexible membrane, the proximally directed force to a force sensor.

### CROSS-REFERENCE TO RELATED APPLICATION

The present application claims priority to U.S. Provisional Application Ser. No. 63/338,662, entitled “Syringe Pressure Sensor Assembly”, filed May 5, 2022, the entire disclosure of which is hereby incorporated by reference in its entirety.

### BACKGROUND

### 1. Field

The present disclosure relates generally to syringes and, in some non-limiting embodiments or aspects, to a low cost high accuracy syringe pressure sensor assembly.

### 2. Technical Considerations

There is an increasing need for syringe pumps due to new medications that are required to be delivered with higher flow consistency and accuracy. However, existing syringe pump systems may have poor pressure sensing performance.

Pressure sensing performance is safety feature that prevents or reduces over-pressure of an IV line and/or a vein of a patient if the IV line becomes occluded (e.g., due to flow restriction, due to blockage, etc.). In addition, high accuracy pressure sensing is desired in order to provide for more accurate early detection of IV line occlusion (e.g., due to an obstruction or partial blockage, etc.).

Existing syringe pumps may sense syringe pressure indirectly by measuring a force that a pump actuator exerts on a plunger rod of a syringe and back calculating a syringe fluid pressure by multiplying the indirect force measurement by a frontal section area of a syringe barrel of the syringe and/or a value read from a syringe identifier label. However, this existing approach to pressure sensing has inherent inaccuracies due to a sliding friction force that exists between a stopper and the syringe barrel. This sliding friction can vary by a significant percentage (e.g., up to 50%, etc.) between syringe manufacturing lots due to a number of factors such as, manufacturing tolerances, syringe barrel lubrication area dispersal pattern, and/or the like. In addition, due to barrel taper, which is used for injection molding, syringe friction typically varies by about 10% as the stopper travels down the barrel, and this variation in sliding friction directly affects the calculated syringe pump pressure accuracy.

Existing attempts to address this inaccuracy due to the variable syringe friction primarily involve using a pressure sensing diaphragm that is placed in line with an IV extension tubing that exits the syringe. The diaphragm is typically put into a form fitting cavity with a load cell to measure a diaphragm “ballooning” force that occurs when the IV line is pressurized. The IV line pressure can then be back calculated. However, this diaphragm-based approach is not used extensively in syringe pumps because the diaphragm is expensive to produce due to the diaphragm being made from relatively expensive liquid silicone rubber (LSR) material, which is also expensive to mold.

### SUMMARY

Accordingly, provided are improved systems, devices, products, apparatus, assemblies, and/or methods for syringe pressure sensors. For example, non-limiting embodiments or aspects of the present disclosure may provide a syringe pressure sensor assembly that improves syringe pump pressure sensing and/or reduces a cost of disposables or consumables used for sensing syringe pressure.

According to some non-limiting embodiments or aspects, provided is a syringe pressure sensor assembly, including: a syringe barrel; a plunger rod including an outer support structure and an inner force translation rod; a stopper secured to the plunger rod, wherein the stopper is axially movable within the syringe barrel, wherein the stopper includes a flexible membrane including a proximal face and a distal face opposite the proximal face and a sidewall surrounding the flexible membrane, wherein the sidewall of the stopper is in fluid tight engagement with an inner surface of the syringe barrel; and wherein the outer support structure is secured to the sidewall of the stopper, wherein the inner force translation rod contacts the proximal face of the flexible membrane, wherein the flexible membrane moves proximally relative to the sidewall in response to a proximally directed force applied to the distal face of the flexible membrane, and wherein the inner force translation rod transfers, in response to the proximal movement of the flexible membrane, the proximally directed force to a force sensor.

In some non-limiting embodiments or aspects, the force sensor is integrated with the inner force translation rod.

In some non-limiting embodiments or aspects, the inner force translation rod extends between a proximal end and a distal end, wherein the distal end of the inner force translation rod contacts the proximal face of the flexible membrane, and wherein the proximal end of the inner force translation rod contacts the force sensor.

In some non-limiting embodiments or aspects, the outer support structure includes an inner cavity, wherein the inner force translation rod extends within the inner cavity of the outer support structure.

In some non-limiting embodiments or aspects, the force sensor includes at least one of the following: a load cell, a carbon powder-based force sensor, a strain gauge, a piezoelectric sensor, or any combination thereof.

In some non-limiting embodiments or aspects, the sidewall of the flexible membrane includes at least one protrusion extending radially inward from the sidewall, and wherein a distal end of the outer support structure includes at least one indentation configured to receive the at least one protrusion, and wherein the stopper is secured to the outer support structure via a complementary mating of the at least one protrusion and the at least one indentation.

In some non-limiting embodiments or aspects, a first portion of the flexible membrane has a first thickness between the proximal face and the distal face, wherein the first portion of the flexible membrane is surrounded a second portion of the flexible membrane, wherein the second portion of the flexible membrane has a second thickness between the proximal face and the distal face that is less than the first thickness of the first portion, wherein a third portion of the flexible membrane surrounds the first portion and the second portion, wherein the proximal face of the flexible membrane at the third portion thereof is in contact with a rigid stopper core that inhibits the proximal movement of the flexible membrane, and wherein the inner force translation rode contacts the proximal face of the flexible membrane at the first portion of the flexible membrane.

In some non-limiting embodiments or aspects, the flexible membrane has one of a dome shape, a conical shape, and a frusto-conical shape, and wherein the proximal face of the flexible membrane is spaced apart from a distal end of the outer support structure.

In some non-limiting embodiments or aspects, the syringe pressure sensor assembly further includes a syringe actuator assembly configured to apply a distal force to a proximal end of the plunger rod.

In some non-limiting embodiments or aspects, the syringe actuator assembly includes an actuator base and an actuator slider that moves linearly relative to the actuator base, wherein the actuator base includes a track, wherein the actuator slider includes a rail that is received within the track and a plunger contact that extends from the rail and contacts the plunger to apply the distal force to the proximal end of the plunger rod in response to the linear movement of the actuator slider relative to the actuator base.

In some non-limiting embodiments or aspects, the plunger contact of the actuator slider includes the force sensor.

According to some non-limiting embodiments or aspects, provided is a system, including: a syringe pressure sensor assembly, including: a syringe barrel; a plunger rod including an outer support structure and an inner force translation rod; a stopper secured to the plunger rod, wherein the stopper is axially movable within the syringe barrel, wherein the stopper includes a flexible membrane including a proximal face and a distal face opposite the proximal face and a sidewall surrounding the flexible membrane, wherein the sidewall of the stopper is in fluid tight engagement with an inner surface of the syringe barrel; and wherein the outer support structure is secured to the sidewall of the stopper, wherein the inner force translation rod contacts the proximal face of the flexible membrane, wherein the flexible membrane moves proximally relative to the sidewall in response to a proximally directed force applied to the distal face of the flexible membrane, and wherein the inner force translation rod transfers, in response to the proximal movement of the flexible membrane, the proximally directed force to a force sensor; and a syringe pump including a motor and at least one processor programmed and/or configured to: control the motor to move the plunger rod axially relative to the syringe barrel at a known, constant speed; receive, from the force sensor, a measurement of an internal syringe pressure when the plunger rod is moved axially relative to the syringe barrel at the known, constant speed; obtain a flow rate of a fluid in a catheter connected to the syringe barrel when the plunger rod is moved axially relative to the syringe barrel at the known, constant speed; and determine, based on the measurement of the internal syringe pressure and the flow rate, a flow resistance of the catheter.

In some non-limiting embodiments or aspects, the syringe pump further includes an encoder connected to the motor that continuously tracks a rotational movement of the motor, and wherein the flow rate of the fluid in the catheter is determined based on the rotational movement of the motor tracked by the encoder.

In some non-limiting embodiments or aspects, the at least one processor is further programmed and/or configured to: control, based on the flow resistance of the catheter, the motor to move the plunger rod axially relative to the syringe barrel to adjust the flow rate of the fluid to achieve a desired velocity of the fluid in the catheter.

In some non-limiting embodiments or aspects, the system further includes: a syringe actuator assembly configured to apply a distal force to a proximal end of the plunger rod, wherein the at least one processor is programmed and/or configured to control the motor to drive the syringe actuator assembly to move the plunger rod axially relative to the syringe barrel by applying the distal force to the proximal end of the plunger rod.

According to some non-limiting embodiments or aspects, provided is a method, including: providing the syringe pressure sensor assembly; moving, with a syringe pump, the plunger rod axially relative to the syringe barrel at a known, constant speed; measuring, with the force sensor, an internal syringe pressure when the plunger rod is moved axially relative to the syringe barrel at the known, constant speed; measuring, with the syringe pump, a flow rate of a fluid in a catheter connected to the syringe barrel when the plunger rod is moved axially relative to the syringe barrel at the known, constant speed; and moving, with the syringe pump, based on a flow resistance of the catheter, the plunger rod axially relative to the syringe barrel to adjust the flow rate of the fluid to achieve a desired velocity of the fluid in the catheter, wherein the flow resistance of the catheter is determined based on the measurement of the internal syringe pressure and the flow rate.

In some non-limiting embodiments or aspects, the method further includes: providing a syringe actuator assembly configured to apply a distal force to a proximal end of the plunger rod, wherein syringe pump drives the syringe actuator assembly to move the plunger rod axially relative to the syringe barrel by applying the distal force to the proximal end of the plunger rod.

According to some non-limiting embodiments or aspects, provided is a method, including: controlling, with at least one processor, a syringe pump to move a plunger rod of a syringe axially relative to a syringe barrel of the syringe at a known, constant speed; receiving, with the at least one processor, from a force sensor, an internal syringe pressure measured when the plunger rod is moved axially relative to the syringe barrel at the known, constant speed; obtaining, with the at least one processor, from the syringe pump, a flow rate of a fluid in a catheter connected to the syringe barrel determined when the plunger rod is moved axially relative to the syringe barrel at the known, constant speed; determining, with at least one processor, based on the measurement of the internal syringe pressure and the flow rate, a flow resistance of the catheter; and controlling, with the at least one processor, based on the flow resistance of the catheter, the syringe pump to move the plunger rod axially relative to the syringe barrel to adjust the flow rate of the fluid to achieve a desired velocity of the fluid in the catheter.

In some non-limiting embodiments or aspects, the flow rate of the fluid in the catheter is determined based on a rotational movement of a motor of the syringe pump.

According to some non-limiting embodiments or aspects, provided is a system, including: at least one processor programmed and/or configured to: control a syringe pump to move a plunger rod of a syringe axially relative to a syringe barrel of the syringe at a known, constant speed; receive, from a force sensor, an internal syringe pressure measured when the plunger rod is moved axially relative to the syringe barrel at the known, constant speed; obtain, from the syringe pump, a flow rate of a fluid in a catheter connected to the syringe barrel determined when the plunger rod is moved axially relative to the syringe barrel at the known, constant speed; determine, based on the measurement of the internal syringe pressure and the flow rate, a flow resistance of the catheter; and control, based on the flow resistance of the catheter, the syringe pump to move the plunger rod axially relative to the syringe barrel to adjust the flow rate of the fluid to achieve a desired velocity of the fluid in the catheter.

Further embodiments or aspects are set forth in the following numbered clauses:
Clause 1. A syringe pressure sensor assembly, comprising: a syringe barrel; a plunger rod including an outer support structure and an inner force translation rod; a stopper secured to the plunger rod, wherein the stopper is axially movable within the syringe barrel, wherein the stopper includes a flexible membrane including a proximal face and a distal face opposite the proximal face and a sidewall surrounding the flexible membrane, wherein the sidewall of the stopper is in fluid tight engagement with an inner surface of the syringe barrel; and wherein the outer support structure is secured to the sidewall of the stopper, wherein the inner force translation rod contacts the proximal face of the flexible membrane, wherein the flexible membrane moves proximally relative to the sidewall in response to a proximally directed force applied to the distal face of the flexible membrane, and wherein the inner force translation rod transfers, in response to the proximal movement of the flexible membrane, the proximally directed force to a force sensor.
Clause 2. The syringe pressure sensor assembly of clause 1, wherein the force sensor is integrated with the inner force translation rod.
Clause 3. The syringe pressure sensor assembly of any of clauses 1 and 2, wherein the inner force translation rod extends between a proximal end and a distal end, wherein the distal end of the inner force translation rod contacts the proximal face of the flexible membrane, and wherein the proximal end of the inner force translation rod contacts the force sensor.
Clause 4. The syringe pressure sensor assembly of any of clauses 1-3, wherein the outer support structure includes an inner cavity, wherein the inner force translation rod extends within the inner cavity of the outer support structure.
Clause 5. The syringe pressure sensor assembly of any of clauses 1-4, wherein the force sensor includes at least one of the following: a load cell, a carbon powder-based force sensor, a strain gauge, a piezoelectric sensor, or any combination thereof.
Clause 6. The syringe pressure sensor assembly of any of clauses 1-5, wherein the sidewall of the flexible membrane includes at least one protrusion extending radially inward from the sidewall, and wherein a distal end of the outer support structure includes at least one indentation configured to receive the at least one protrusion, and wherein the stopper is secured to the outer support structure via a complementary mating of the at least one protrusion and the at least one indentation.
Clause 7. The syringe pressure sensor assembly of any of clauses 1-6, wherein a first portion of the flexible membrane has a first thickness between the proximal face and the distal face, wherein the first portion of the flexible membrane is surrounded a second portion of the flexible membrane, wherein the second portion of the flexible membrane has a second thickness between the proximal face and the distal face that is less than the first thickness of the first portion, wherein a third portion of the flexible membrane surrounds the first portion and the second portion, wherein the proximal face of the flexible membrane at the third portion thereof is in contact with a rigid stopper core that inhibits the proximal movement of the flexible membrane, and wherein the inner force translation rode contacts the proximal face of the flexible membrane at the first portion of the flexible membrane.
Clause 8. The syringe pressure sensor assembly of any of clauses 1-7, wherein the flexible membrane has one of a dome shape, a conical shape, and a frusto-conical shape, and wherein the proximal face of the flexible membrane is spaced apart from a distal end of the outer support structure.
Clause 9. The syringe pressure sensor assembly of any of clauses 1-8, further comprising: a syringe actuator assembly configured to apply a distal force to a proximal end of the plunger rod.
Clause 10. The syringe pressure sensor assembly of any of clauses 1-9, wherein the syringe actuator assembly includes an actuator base and an actuator slider that moves linearly relative to the actuator base, wherein the actuator base includes a track, wherein the actuator slider includes a rail that is received within the track and a plunger contact that extends from the rail and contacts the plunger to apply the distal force to the proximal end of the plunger rod in response to the linear movement of the actuator slider relative to the actuator base.
Clause 11. The syringe pressure sensor assembly of any of clauses 1-10, wherein the plunger contact of the actuator slider includes the force sensor.
Clause 12. A system, comprising: the syringe pressure sensor assembly of any of clauses 1-11; and a syringe pump including a motor and at least one processor programmed and/or configured to: control the motor to move the plunger rod axially relative to the syringe barrel at a known, constant speed; receive, from the force sensor, a measurement of an internal syringe pressure when the plunger rod is moved axially relative to the syringe barrel at the known, constant speed; obtain a flow rate of a fluid in a catheter connected to the syringe barrel when the plunger rod is moved axially relative to the syringe barrel at the known, constant speed; and determine, based on the measurement of the internal syringe pressure and the flow rate, a flow resistance of the catheter.
Clause 13. The system of clause 12, wherein the syringe pump further includes an encoder connected to the motor that continuously tracks a rotational movement of the motor, and wherein the flow rate of the fluid in the catheter is determined based on the rotational movement of the motor tracked by the encoder.
Clause 14. The system of any of clauses 12 and 13, wherein the at least one processor is further programmed and/or configured to: control, based on the flow resistance of the catheter, the motor to move the plunger rod axially relative to the syringe barrel to adjust the flow rate of the fluid to achieve a desired velocity of the fluid in the catheter.
Clause 15. The system of any of clauses 12-14, further comprising: a syringe actuator assembly configured to apply a distal force to a proximal end of the plunger rod, wherein the at least one processor is programmed and/or configured to control the motor to drive the syringe actuator assembly to move the plunger rod axially relative to the syringe barrel by applying the distal force to the proximal end of the plunger rod.
Clause 16. A method, comprising: providing the syringe pressure sensor assembly of any of clauses 1-11; moving, with a syringe pump, the plunger rod axially relative to the syringe barrel at a known, constant speed; measuring, with the force sensor, an internal syringe pressure when the plunger rod is moved axially relative to the syringe barrel at the known, constant speed; measuring, with the syringe pump, a flow rate of a fluid in a catheter connected to the syringe barrel when the plunger rod is moved axially relative to the syringe barrel at the known, constant speed; and moving, with the syringe pump, based on a flow resistance of the catheter, the plunger rod axially relative to the syringe barrel to adjust the flow rate of the fluid to achieve a desired velocity of the fluid in the catheter, wherein the flow resistance of the catheter is determined based on the measurement of the internal syringe pressure and the flow rate.
Clause 17. The method of clause 16, further comprising: providing a syringe actuator assembly configured to apply a distal force to a proximal end of the plunger rod, wherein syringe pump drives the syringe actuator assembly to move the plunger rod axially relative to the syringe barrel by applying the distal force to the proximal end of the plunger rod.
Clause 18. A method, comprising: controlling, with at least one processor, a syringe pump to move a plunger rod of a syringe axially relative to a syringe barrel of the syringe at a known, constant speed; receiving, with the at least one processor, from a force sensor, an internal syringe pressure measured when the plunger rod is moved axially relative to the syringe barrel at the known, constant speed; obtaining, with the at least one processor, from the syringe pump, a flow rate of a fluid in a catheter connected to the syringe barrel determined when the plunger rod is moved axially relative to the syringe barrel at the known, constant speed; determining, with at least one processor, based on the measurement of the internal syringe pressure and the flow rate, a flow resistance of the catheter; and controlling, with the at least one processor, based on the flow resistance of the catheter, the syringe pump to move the plunger rod axially relative to the syringe barrel to adjust the flow rate of the fluid to achieve a desired velocity of the fluid in the catheter.
Clause 19. The method of clause 18, wherein the flow rate of the fluid in the catheter is determined based on a rotational movement of a motor of the syringe pump.
Clause 20. A system, comprising: at least one processor programmed and/or configured to: control a syringe pump to move a plunger rod of a syringe axially relative to a syringe barrel of the syringe at a known, constant speed; receive, from a force sensor, an internal syringe pressure measured when the plunger rod is moved axially relative to the syringe barrel at the known, constant speed; obtain, from the syringe pump, a flow rate of a fluid in a catheter connected to the syringe barrel determined when the plunger rod is moved axially relative to the syringe barrel at the known, constant speed; determine, based on the measurement of the internal syringe pressure and the flow rate, a flow resistance of the catheter; and control, based on the flow resistance of the catheter, the syringe pump to move the plunger rod axially relative to the syringe barrel to adjust the flow rate of the fluid to achieve a desired velocity of the fluid in the catheter.

These and other features and characteristics of the present disclosure, as well as the methods of operation and functions of the related elements of structures and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of limits. As used in the specification and the claims, the singular form of “a,” “an,” and “the” include plural referents unless the context clearly dictates otherwise.

### BRIEF DESCRIPTION OF THE DRAWINGS

Additional advantages and details of embodiments or aspects of the present disclosure are explained in greater detail below with reference to the exemplary embodiments that are illustrated in the accompanying schematic figures, in which:

FIG. 1A is a sectional view of non-limiting embodiments or aspects of a syringe pressure sensor assembly;

FIG. 1B is perspective view of non-limiting embodiments or aspects of a syringe pressure sensor assembly;

FIG. 1C is a perspective view of non-limiting embodiments or aspects of a syringe pressure sensor assembly including a shortened plunger rod;

FIGS. 2A-2C are perspective view of non-limiting embodiments or aspects of a stopper;

FIGS. 3A and 3B are perspective views of non-limiting embodiments or aspects of a shortened plunger rod;

FIG. 4 is a perspective view of non-limiting embodiments or aspects of an outer support structure of a plunger rod.

FIG. 5 is a perspective view of non-limiting embodiments or aspects of a force sensor;

FIG. 6 is a sectional view of non-limiting embodiments or aspects of a syringe pressure sensor assembly;

FIG. 7A is a perspective view of non-limiting embodiments or aspects of an actuator base of a syringe pump actuator assembly;

FIG. 7B is a perspective view of non-limiting embodiments or aspects of an actuator slider of a syringe pump actuator assembly;

FIG. 8 is a diagram of non-limiting embodiments or aspects of an environment in which systems, devices, products, apparatus, assemblies, and/or methods, described herein, may be implemented;

FIG. 9 is a diagram of non-limiting embodiments or aspects of components of a syringe pump;

FIG. 10 is a diagram of non-limiting embodiments or aspects of components of one or more devices and/or one or more systems of FIG. 9; and

FIG. 11 is a flowchart of non-limiting embodiments or aspects of a process for delivering fluid from a syringe.

### DETAILED DESCRIPTION

It is to be understood that the present disclosure may assume various alternative variations and step sequences, except where expressly specified to the contrary. It is also to be understood that the specific devices and processes illustrated in the attached drawings, and described in the following specification, are simply exemplary and non-limiting embodiments or aspects. Hence, specific dimensions and other physical characteristics related to the embodiments or aspects disclosed herein are not to be considered as limiting.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary aspects of the invention. Hence, specific dimensions and other physical characteristics related to the aspects disclosed herein are not to be considered as limiting. All numbers and ranges used in the specification and claims are to be understood as being modified in all instances by the term “about”. By “about” is meant plus or minus twenty-five percent of the stated value, such as plus or minus ten percent of the stated value. However, this should not be considered as limiting to any analysis of the values under the doctrine of equivalents.

Unless otherwise indicated, all ranges or ratios disclosed herein are to be understood to encompass the beginning and ending values and any and all subranges or sub-ratios subsumed therein. For example, a stated range or ratio of “1 to 10” should be considered to include any and all subranges or sub-ratios between (and inclusive of) the minimum value of 1 and the maximum value of 10; that is, all subranges or sub-ratios beginning with a minimum value of 1 or more and ending with a maximum value of 10 or less. The ranges and/or ratios disclosed herein represent the average values over the specified range and/or ratio.

The terms “first”, “second”, and the like are not intended to refer to any particular order or chronology, but refer to different conditions, properties, or elements.

No aspect, component, element, structure, act, step, function, instruction, and/or the like used herein should be construed as critical or essential unless explicitly described as such. Also, as used herein, the articles “a” and “an” are intended to include one or more items, and may be used interchangeably with “one or more” and “at least one.” Furthermore, as used herein, the term “set” is intended to include one or more items (e.g., related items, unrelated items, a combination of related and unrelated items, etc.) and may be used interchangeably with “one or more” or “at least one.” Where only one item is intended, the term “one” or similar language is used. Also, as used herein, the terms “has,” “have,” “having,” or the like are intended to be open-ended terms. Further, the phrase “based on” is intended to mean “based at least in partially on” unless explicitly stated otherwise.

As used herein, the terms “communication” and “communicate” refer to the receipt or transfer of one or more signals, messages, commands, or other type of data. For one unit (e.g., any device, system, or component thereof) to be in communication with another unit means that the one unit is able to directly or indirectly receive data from and/or transmit data to the other unit. This may refer to a direct or indirect connection that is wired and/or wireless in nature. Additionally, two units may be in communication with each other even though the data transmitted may be modified, processed, relayed, and/or routed between the first and second unit. For example, a first unit may be in communication with a second unit even though the first unit passively receives data and does not actively transmit data to the second unit. As another example, a first unit may be in communication with a second unit if an intermediary unit processes data from one unit and transmits processed data to the second unit. It will be appreciated that numerous other arrangements are possible.

It will be apparent that systems and/or methods, described herein, can be implemented in different forms of hardware, software, or a combination of hardware and software. The actual specialized control hardware or software code used to implement these systems and/or methods is not limiting of the implementations. Thus, the operation and behavior of the systems and/or methods are described herein without reference to specific software code, it being understood that software and hardware can be designed to implement the systems and/or methods based on the description herein.

Some non-limiting embodiments or aspects are described herein in connection with thresholds. As used herein, satisfying a threshold may refer to a value being greater than the threshold, more than the threshold, higher than the threshold, greater than or equal to the threshold, less than the threshold, fewer than the threshold, lower than the threshold, less than or equal to the threshold, equal to the threshold, etc.

As used herein, the term “computing device” or “computer device” may refer to one or more electronic devices that are configured to directly or indirectly communicate with or over one or more networks. The computing device may be a mobile device, a desktop computer, or the like. Furthermore, the term “computer” may refer to any computing device that includes the necessary components to receive, process, and output data, and normally includes a display, a processor, a memory, an input device, and a network interface. An “application” or “application program interface” (API) refers to computer code or other data sorted on a computer-readable medium that may be executed by a processor to facilitate the interaction between software components, such as a client-side front-end and/or server-side back-end for receiving data from the client. An “interface” refers to a generated display, such as one or more graphical user interfaces (GUIs) with which a user may interact, either directly or indirectly (e.g., through a keyboard, mouse, touchscreen, etc.).

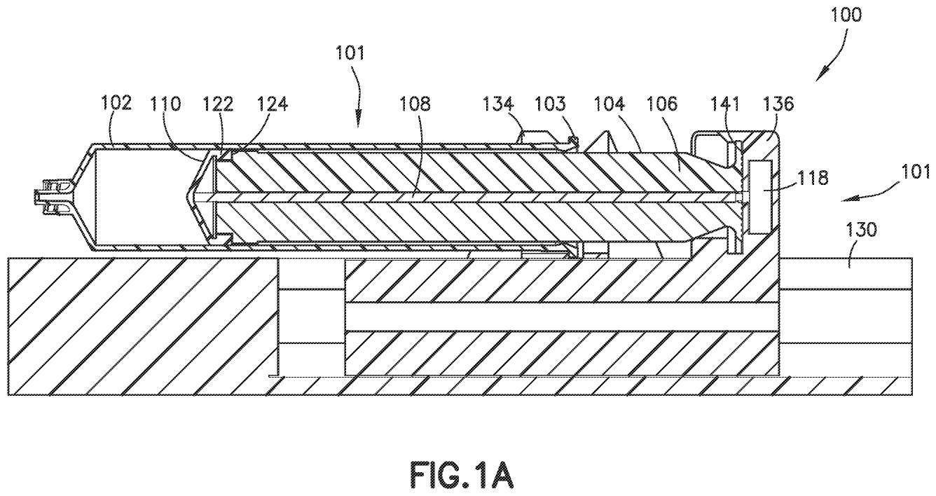

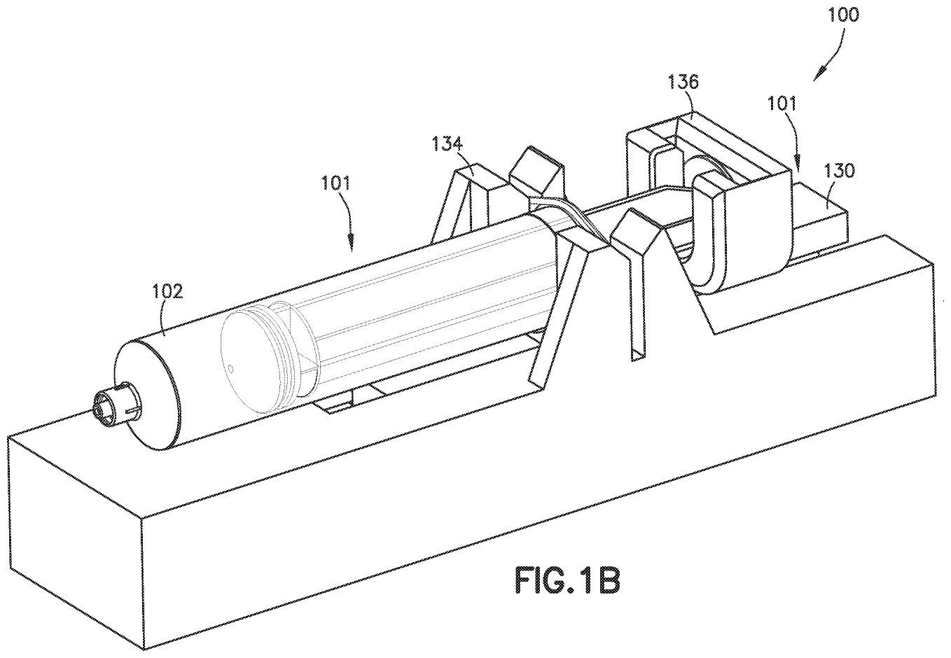

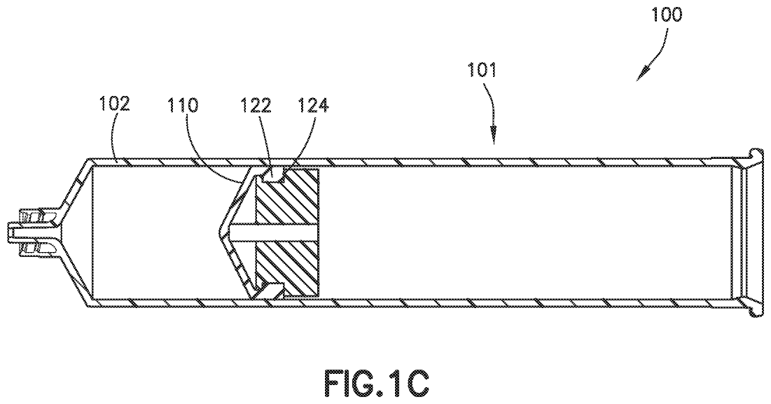

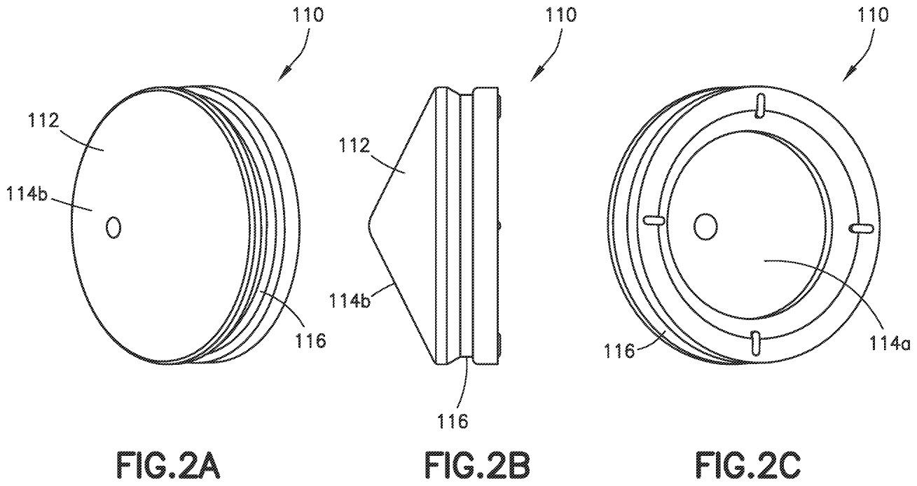

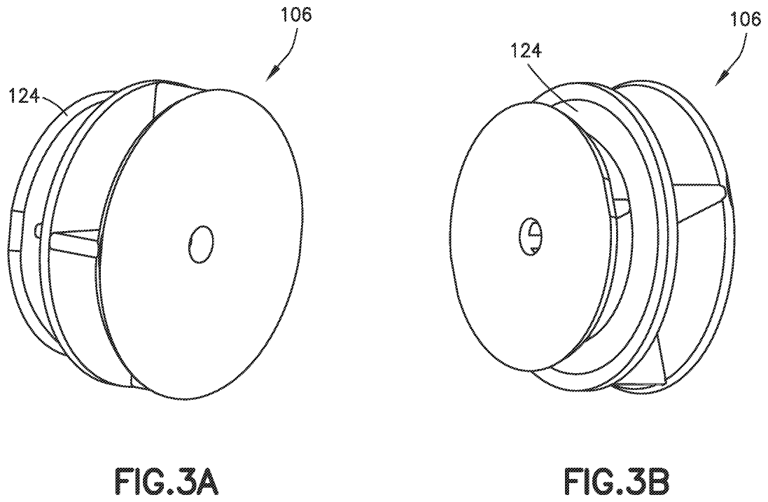

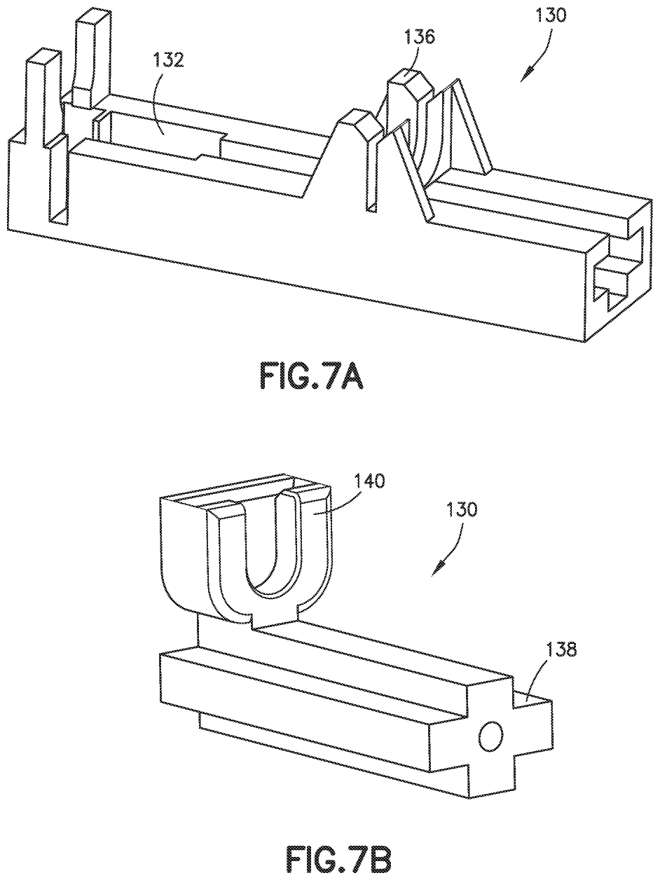

Referring to FIGS. 1A-1C, 2A-2C, 3A, 3B, 4-6, 7A, and 7B, syringe pressure assembly 100 may include syringe 101, syringe actuator assembly 129, and/or force sensor 118.

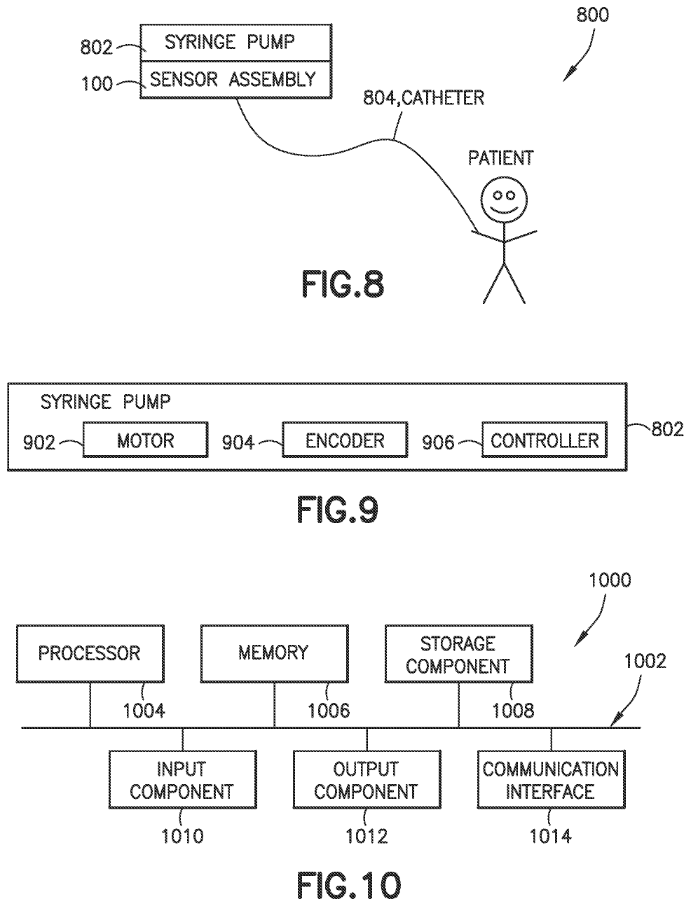

Syringe 101 may include syringe barrel 102, plunger rod 104, and/or stopper 110. Syringe barrel 102 may extend between a proximal end including a proximal opening configured to receive stopper 110 and/or plunger rod 104 and a distal end including a distal opening (e.g., a luer lock opening, etc.) configured to be connected to a catheter (e.g., catheter 804 described herein below with respect to FIG. 8, etc.) and through which a fluid can be expelled from syringe 101. The proximal end of syringe barrel 102 may include flange 103 extending radially outward from syringe barrel 102.

Plunger rod 104 may include outer support structure 106 and inner force translation rod 108. Outer support structure 106 may extend between a proximal end and a distal end. Inner force translation rod 108 may extend between a proximal end and a distal end. Referring specifically to FIG. 1C, in some non-limiting embodiments or aspects, plunger rod 104 may be significantly shorter for more advantageous use with smaller length syringe pumps.

Stopper 110 may be secured to plunger rod 104, and stopper 110 and/or plunger rod 104 may be axially moveable within syringe barrel 102. For example, stopper 110 may be secured to the distal end of outer support structure 106. Stopper 110 may include flexible membrane 112 including proximal face 114a and distal face 114b opposite proximal face 114a and sidewall 116 surrounding flexible membrane 112. Sidewall 116 of stopper 110 may be in fluid tight engagement with an inner surface of syringe barrel 102. Outer support structure 106 may be secured to sidewall 116 of stopper 110. For example, sidewall 116 of flexible membrane 112 may include at least one protrusion 122 extending radially inward from sidewall 116 and the distal end of outer support structure 106 may include at least one indentation 124 configured to receive the at least one protrusion 122 (and/or vice-versa). As an example, stopper 110 may be secured to outer support 106 structure via a complementary mating of the at least one protrusion 122 and the at least one indentation 124. In some non-limiting embodiments or aspects, proximal face 114a (e.g., at least a portion of proximal face 114a, etc.) of flexible membrane 112 is spaced apart from the distal end of the outer support structure 106.

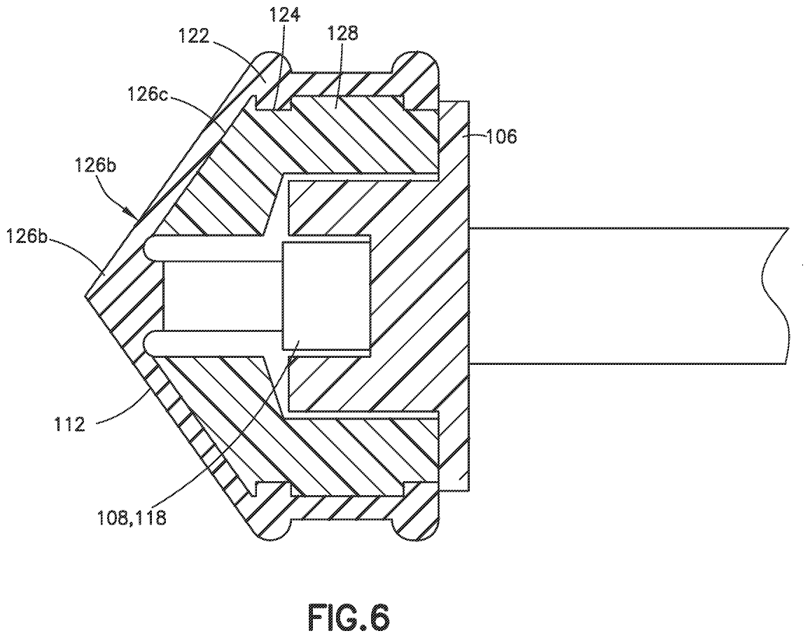

Referring specifically to FIG. 6, in some non-limiting embodiments or aspects, a first portion 126a of flexible membrane 112 has a first thickness between proximal face 114a and distal face 114b, and the first portion 126a of flexible membrane 112 is surrounded a second portion 126b of flexible membrane 112 that has a second thickness between proximal face 114a and distal face 114b that is less than the first thickness of the first portion 126a. In such an example, a third portion 126c of flexible membrane 112 may surround the first portion 126a and the second portion 126b, and proximal face 114a of flexible membrane 112 at the third portion 126c thereof may in contact with rigid stopper core 128 that inhibits the proximal movement of flexible membrane 112 (e.g., at the third portion 126c thereof, etc.), and inner force translation rod 108 (e.g., force sensor 118 implemented as inner force translation rod 108, etc.) may contact (e.g., directly contact, etc.) proximal face 114a of flexible membrane 112 at the first portion 126a of flexible membrane 112. In this way, the difference in thickness between the first portion 126a and the second portion 126 may improve the free axial movement of a portion of the flexible membrane 112 connected to and/or in contact with the force sensor 118 by acting as a hinge, which enables reducing a restriction of movement and obtaining a more accurate force measurement. For example, if the flexible membrane 112 has an even thickness, the axial movement of the portion of the flexible membrane 112 connected to and/or in contact with the force sensor 118 may be reduced or limited due to an internal stiffness of the flexible membrane.

Inner force translation rod 108 may contact (e.g., directly contact, etc.) proximal face 114a of flexible membrane 112. For example, the distal end of inner force transaction rod may contact proximal face 114a of flexible membrane 112. Flexible membrane 112 may move proximally relative to sidewall 116 of stopper 110 in response to a proximally directed force applied to distal face 114b of flexible membrane 112, and inner force translation rod 108 may transfer, in response to the proximal movement of flexible membrane 112, the proximally directed force to force sensor 118, which may be in contact (e.g., direct contact, etc.) with the proximal end of inner force transaction rod 108. For example, flexible membrane 112 may move in accordance with fluid and/or air pressure changes within syringe barrel 102. As an example, flexible membrane 112 may have one of a dome shape, a conical shape, and a frusto-conical shape, and the one of the dome shape, the conical shape, and the frusto-conical shape may elastically deform in an axis parallel with the axial direction of syringe barrel 102 in response to pressure changes occurring in syringe barrel 102, and inner force translation rod 108 may contact and/or support a center of flexible membrane 112 such that the elastic deformation forces of flexible membrane 112 are transferred to inner force translation rod 108 (and/or via inner for translation rod 108 to force sensor 118).

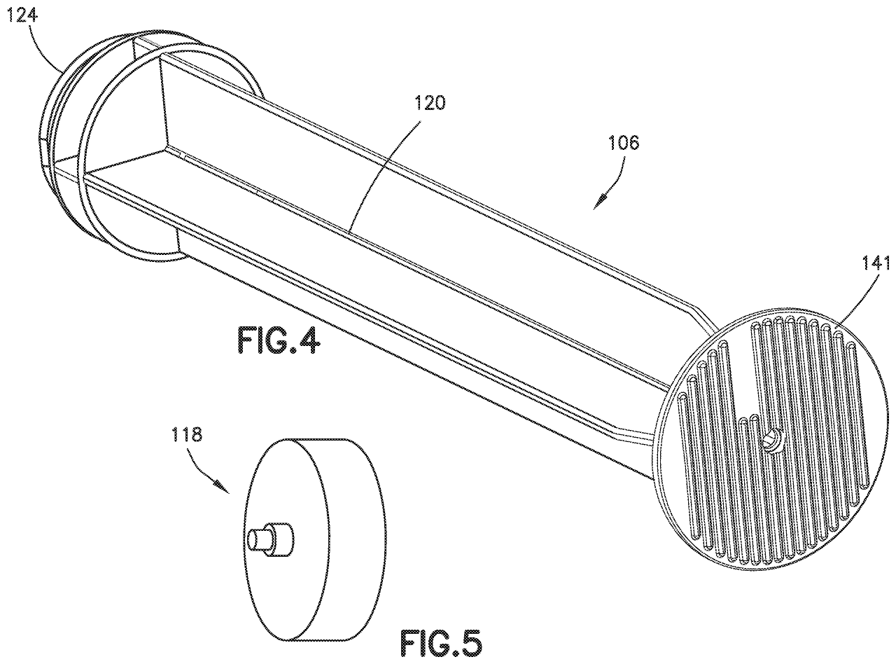

Referring specifically to FIG. 4, in some non-limiting embodiments or aspects, outer support structure 106 includes inner cavity 120. For example, inner force translation rod 108 may extend with inner cavity 120 of outer support structure 106.

Force sensor 118 may include at least one of the following: a load cell, a carbon powder-based force sensor, a strain gauge, a piezoelectric sensor, or any combination thereof. Referring specifically to FIG. 6, in some non-limiting embodiments or aspects, force sensor 118 is integrated with inner force translation rod 108. For example, inner force translation rod 108 may include a force transducer load cell. Referring specifically to FIGS. 1A-1C, in some non-limiting embodiments or aspects, the distal end of inner force translation rod 108 contacts (e.g., directly contacts, etc.) proximal face 114a of flexible membrane 112 and the proximal end of inner for transaction rods contacts (e.g., directly contacts, etc.) and/or is secured to force sensor 118. For example, inner force translation rod 108 may include a column that contacts and/or is secured to a force transducer load cell.

Accordingly, as illustrated by the arrows in FIGS. 1A-1C and 6, forces due to sliding friction between stopper 110 and the inner surface of syringe barrel 102 may be transferred to outer support structure 106 without significantly affecting the elastic deformation of at a center of flexible membrane 112 of stopper 110 (e.g., at the first portion 126a and/or second portion 126b of flexible membrane 112, etc.). For example, by supporting stopper 110 at sidewall 116 thereof (e.g., only at sidewall 116, only at sidewall 116 and/or via rigid stopper core 128, etc.) with outer support structure 106, the forces due to the sliding friction between stopper 110 and the inner surface of syringe barrel 102 may be transferred to directly to outer support structure 106 (e.g., solely to outer support structure 106, primarily to outer support structure 106, etc.), and an inner force due to pressure changes within syringe barrel 102 may be transferred directly to inner force translation rod 108 (e.g., solely to inner force translation rod 108, primarily to inner force translation rod 108, etc.). Further, because the syringe pressure forces may be translated to force sensor 118 significantly independent from the syringe frictional forces received by outer support structure 106, syringe pressure may be back calculated based on measurements of force sensor 118 without significant error due to the sliding frictional forces.

Syringe actuator assembly 129 may apply a distal force to the proximal end of plunger rod 104. Syringe actuator assembly 129 may include actuator base 130 and actuator slider 136 that moves linearly relative to actuator base 130. Actuator base 130 may include track 132 and flange holder 134 configured to receive flange 103 of syringe barrel 102, and actuator slider 136 may include rail 138 that is received and slides within track 132 and a plunger contact 140 that extends from the rail in a direction perpendicular to the sliding direction and contacts plunger rod 104 to apply the distal force to the proximal end of plunger rod 104 in response to the linear movement of actuator slider 136 relative to actuator base 130 as flange holder 134 holds syringe barrel 102 stationary by inhibiting or preventing a corresponding linear movement thereof.

Referring specifically to FIG. 1A, in some non-limiting embodiments or aspects, actuator slider contact surface 140 of actuator slider 136 may include force sensor 118. For example, force sensor 118 may be included within actuator slider 136 such that force sensor 118 moves with plunger rod 104 when actuator slider contact surface 140 applies the distally directed force to the proximal end of plunger rod 104. As an example, actuator slider contact surface 140 may contact (e.g., directly contact, etc.) plunger rod thumb flange 141 at the proximal end of plunger rod 104.

Accordingly, syringe fluid pressure forces may be translated from stopper 110, through inner force translation rod 108, to force sensor 118, and to actuator slider 136, significantly independent from syringe frictional forces from stopper 110. The frictional forces from the interaction between the stopper and the syringe barrel 102 may be translated from stopper 110, through plunger rod 104, to plunger rod thumb flange 141, to actuator slider contact surface 140, to actuator slider 136, and to a syringe pump (e.g., syringe pump 802, etc.) by outer support structure 106, which enables syringe pressure to be back calculated based on measurements of force sensor 118 without significant error due to the sliding frictional forces.

Non-limiting embodiments or aspects of the present disclosure provide a syringe pressure sensor assembly that is less costly to produce in comparison to using a separate diaphragm-type of pressure sensor, because a syringe stopper is already an existing component in a syringe. Syringe stoppers may also be about 60% of a total cost of a syringe assembly and, therefore, costs of stoppers, which may be sunk costs, are already amortized, and making the stopper dual purpose (for pressure sensing) may not add significantly to an overall cost of the syringe.

Referring now to FIG. 8, FIG. 8 is a diagram of an example environment 800 in which systems, methods, products, apparatuses, assemblies, and/or devices described herein, may be implemented. As shown in FIG. 8, environment 800 may include syringe pressure sensor assembly 100, syringe pump 802, and/or catheter 804.

Referring also to FIG. 9, syringe pump 802 may include motor 902, encoder 904, and/or controller 906. Controller 906 may be programmed and/or configured to control motor 902 to move plunger rod 104 axially relative to syringe barrel 102. For example, controller 906 may control motor 902 to drive syringe actuator assembly 129 to move plunger rod 104 axially relative to syringe barrel 102. In some non-limiting embodiments or aspects, controller 906 may include a processor, a low power microcontroller unit (MCU), and/or the like. Syringe pump 802 may be in wired and/or wireless communication with force sensor 118 and constantly monitor an internal pressure in syringe barrel 102 via measurements received from force sensor 118.

Encoder 904 may be directly connected to a motor axle of motor 902 and continuously track and/or record a rotational movement of the motor axle, which may correspond to an absolute position of plunger rod 104. The rotational movement and/or absolute position of plunger rod 104 may be provided to controller 906, and controller 906 may control a speed of motor 902 to deliver or inject a predefined amount of a fluid. For example, controller 906 may implement a proportional-integral-derivative (PID) controller to control a dosing rate (e.g., a flow rate, etc.) and a dosing amount (e.g., a volume of fluid delivered, etc.) over different dosing patterns (e.g., fluid deliveries, etc.).

Catheter 204 may include a lumen connected at a proximal end to the distal end of syringe barrel 102 and at a distal end to a patient.

Referring now to FIG. 10, FIG. 10 is a diagram of example components of a device 1000. Device 1000 can correspond to one or more devices of syringe pump 802 and/or syringe pressure sensor assembly 100. In some non-limiting embodiments or aspects, one or more devices of syringe pump 802 and/or syringe pressure sensor assembly 100 can include at least one device 1000 and/or at least one component of device 1000. As shown in FIG. 10, device 1000 includes bus 1002, processor 1004, memory 1006, storage component 1008, input component 1010, output component 1012, and communication interface 1014.

Bus 1002 includes a component that permits communication among the components of device 1000. In some non-limiting embodiments or aspects, processor 1004 is implemented in hardware, firmware, or a combination of hardware and software. For example, processor 1004 includes a processor (e.g., a central processing unit (CPU), a graphics processing unit (GPU), an accelerated processing unit (APU), etc.), a microprocessor, a digital signal processor (DSP), and/or any processing component (e.g., a field-programmable gate array (FPGA), an application-specific integrated circuit (ASIC), etc.) that can be programmed to perform a function.

Memory 1006 includes a random access memory (RAM), a read only memory (ROM), and/or another type of dynamic or static storage device (e.g., flash memory, magnetic memory, optical memory, etc.) that stores information and/or instructions for use by processor 1004.

Storage component 1008 stores information and/or software related to the operation and use of device 1000. For example, storage component 1008 includes a hard disk (e.g., a magnetic disk, an optical disk, a magneto-optic disk, a solid state disk, etc.), a compact disc (CD), a digital versatile disc (DVD), a floppy disk, a cartridge, a magnetic tape, and/or another type of computer-readable medium, along with a corresponding drive.

Input component 1010 includes a component that permits device 1000 to receive information, such as via user input (e.g., a touch screen display, a keyboard, a keypad, a mouse, a button, a switch, a microphone, etc.). Additionally, or alternatively, input component 1010 includes a sensor for sensing information (e.g., a global positioning system (GPS) component, an accelerometer, a gyroscope, an actuator, etc.). Output component 1012 includes a component that provides output information from device 1000 (e.g., a display, a speaker, one or more light-emitting diodes (LEDs), etc.).

Communication interface 1014 includes a transceiver-like component (e.g., a transceiver, a separate receiver and transmitter, etc.) that enables device 1000 to communicate with other devices, such as via a wired connection, a wireless connection, or a combination of wired and wireless connections. Communication interface 1014 can permit device 1000 to receive information from another device and/or provide information to another device. For example, communication interface 1014 includes an Ethernet interface, an optical interface, a coaxial interface, an infrared interface, a radio frequency (RF) interface, a universal serial bus (USB) interface, a Wi-Fi interface, a cellular network interface, and/or the like.

Device 1000 can perform one or more processes described herein. Device 1000 can perform these processes based on processor 1004 executing software instructions stored by a computer-readable medium, such as memory 1006 and/or storage component 1008. A computer-readable medium (e.g., a non-transitory computer-readable medium) is defined herein as a non-transitory memory device. A memory device includes memory space located inside of a single physical storage device or memory space spread across multiple physical storage devices.

Software instructions can be read into memory 1006 and/or storage component 1008 from another computer-readable medium or from another device via communication interface 1014. When executed, software instructions stored in memory 1006 and/or storage component 1008 cause processor 1004 to perform one or more processes described herein. Additionally, or alternatively, hardwired circuitry can be used in place of or in combination with software instructions to perform one or more processes described herein. Thus, embodiments described herein are not limited to any specific combination of hardware circuitry and software.

The number and arrangement of components shown in FIG. 10 are provided as an example. In some non-limiting embodiments or aspects, device 1000 includes additional components, fewer components, different components, or differently arranged components than those shown in FIG. 10. Additionally, or alternatively, a set of components (e.g., one or more components) of device 1000 can perform one or more functions described as being performed by another set of components of device 1000.

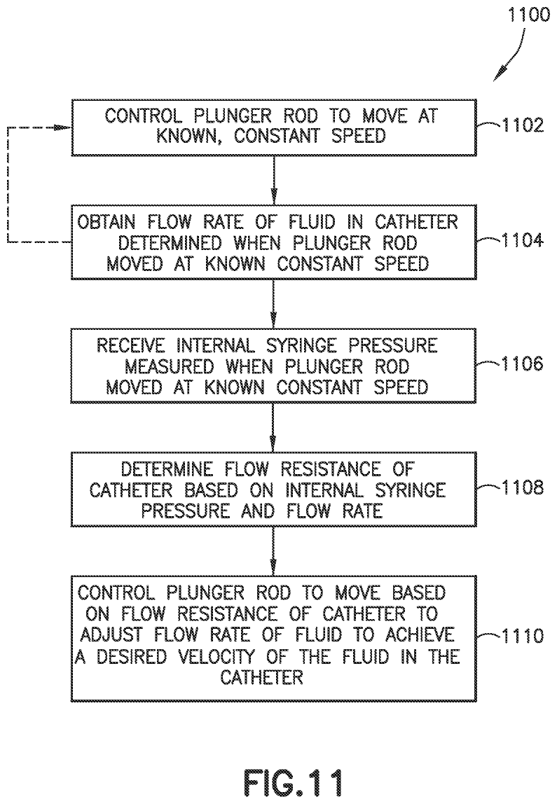

Referring now to FIG. 11, FIG. 11 is a flowchart of non-limiting embodiments or aspects of a process 1100 for delivering fluid from a syringe. In some non-limiting embodiments or aspects, one or more of the steps of process 1100 may be performed (e.g., completely, partially, etc.) by syringe pump 802 (e.g., one or more devices of a system of syringe pump 802, etc.). In some non-limiting embodiments or aspects, one or more of the steps of process 800 may be performed (e.g., completely, partially, etc.) by another device or a group of devices separate from or including syringe pump 802, such as syringe pressure sensor assembly 100 and/or a computing system in communication with syringe pump 802 (e.g., one or more devices of a computing system in communication with syringe pump 802).

As shown in FIG. 11, at step 1102, process 1100 includes controlling a plunger rod to move at a known, constant speed. For example, controller 906 may control syringe pump 802 move plunger rod 104 of syringe 101 axially relative to syringe barrel 102 of syringe 101 at a known, constant speed. As an example, syringe pump 802 may move plunger rod 104 axially relative to syringe barrel 102 at a known, constant speed. For example, controller 906 may control motor 902 to move plunger rod 104 axially relative to syringe barrel 102 at the known, constant speed. In such an example, controller 906 may control motor 902 to drive syringe actuator assembly 129 to move plunger rod 104 axially relative to syringe barrel 102 by applying a distally directed force to the proximal end of plunger rod 104.

As shown in FIG. 11, at step 1104, process 1100 includes obtaining a flow rate of a fluid in a catheter determined when a plunger rod is moved at a known constant speed. For example, controller 906 may obtain, from syringe pump 802, a flow rate of a fluid in catheter 804 connected to syringe barrel 102 that is determined or measured when plunger rod 104 is moved axially relative to syringe barrel 102 at the known, constant speed. As an example, syringe pump 802 may measure a flow rate of a fluid in catheter 804 connected to syringe barrel 102 when plunger rod 104 is moved axially relative to the syringe barrel 102 at the known, constant speed. In such an example, the flow rate of the fluid in catheter 804 may be determined based on a rotational movement of motor 902 tracked by encoder 904.

In such an example, syringe pump 802 may measure a flow rate of a fluid in catheter 804 connected to syringe barrel 102 when plunger rod 104 is moved axially relative to the syringe barrel 102 at at least two different known, constant speeds. For example, syringe pump 802 may measure a first flow rate of a fluid in catheter 804 connected to syringe barrel 102 when plunger rod 104 is moved axially relative to the syringe barrel 102 at a first known, constant speed, and syringe pump 802 may measure a second flow rate of the fluid in catheter 804 connected to syringe barrel 102 when plunger rod 104 is moved axially relative to the syringe barrel 102 at a second known, constant speed different than the first known constant speed.

As previously noted, in this way, as part of a first manually activated flush sequence, reference measurements can be taken automatically after syringe pump 802 is connected to a patient line set-up including catheter 804. For the first manually activated flush sequence, it may be assumed that the flow line is patent and not compromised. Accordingly, syringe pump 802 may calculate and implement, based on the reference measurements, improved or optimal dose or fluid delivery parameters (e.g., flow resistance, flow rate, flow velocity, catheter diameter, etc.), and a calibration flush/infusion may include at least two different volumetric flow levels. For example, reference measurements can be taken while only varying the known flow rate, and the remaining unknown factors in Equations (1) and (2) defined herein below may be calculated or estimated more accurately.

As shown in FIG. 11, at step 1106, process 1100 includes receiving an internal syringe pressure measured when a plunger rod is moved at a known, constant speed. For example, controller 906 may receive, from force sensor 118, an internal syringe pressure (e.g., a pressure between the distal end of syringe barrel 102 and distal face 114b of flexible membrane 112, etc.) this is measured when plunger rod 104 is moved axially relative to syringe barrel 102 at the known, constant speed. As an example, syringe pump 802 may receive, from force sensor 118, an internal syringe pressure (e.g., a pressure between the distal end of syringe barrel 102 and distal face 114b of flexible membrane 112, etc.) this is measured when plunger rod 104 is moved axially relative to syringe barrel 102 at the known, constant speed. In such an example, force sensor 118 may measure the internal syringe pressure when plunger rod 104 is moved axially relative to syringe barrel 102 at the known, constant speed.

In this way, as part of a first manually activated flush sequence, reference measurements can be taken automatically after syringe pump 802 is connected to a patient line set-up including catheter 804. For the first manually activated flush sequence, it may be assumed that the flow line is patent and not compromised. Accordingly, syringe pump 802 may calculate and implement, based on the reference measurements, improved or optimal dose or fluid delivery parameters (e.g., flow resistance, flow rate, flow velocity, catheter diameter, etc.)

As shown in FIG. 11, at step 1108, process 1100 includes determining a flow resistance of a catheter based on an internal syringe pressure and a flow rate of a fluid in the catheter. For example, controller 906 may determine, based on the measurement of the internal syringe pressure and the flow rate, a flow resistance of catheter 804. As an example, to provide an improved or optimal flow velocity and, thereby, improved or optimal line cleaning, syringe pump 802 may detect and process the measurements or data relating to infusion pressure and flow rate, which are closely related to the flow resistance, which depends on catheter geometry. In such an example, because the flow resistance is linked strongly to catheter diameter (e.g., by the fourth power, etc.), the catheter diameter can be assessed if the flow rate, which may be determined using encoder 904, and delta pressure (e.g., determined using force sensor 118, etc.) are known. Accordingly, syringe pump 802 may use these measurements and determinations to adjust the flow rate (e.g., typically decrease the flow rate from a set point, etc.) so that an IV line pressure safety threshold is not violated, which may be accomplished by using a pump algorithm feedback control loop that runs at a high frequency (e.g., a frequency of several hundred or more measurements and control cycles per second, etc.). The pump algorithm may operate by comparing the measured IV Line pressure to the IV line safety threshold. Under normal operation, the pump algorithm may control syringe pump 802 to advance the actuator slider 136 at a target infusion rate set point. However, if the IV Line becomes occluded or partially blocked, the IV line pressure may increase due to hydraulic action, which may cause the IV Line pressure to increase until reaching the IV line pressure safety threshold, at which point the pump algorithm may pause the advancement of the actuator slider until the measured IV line pressure no longer violates the IV line pressure safety threshold, returning to the infusion rate target setting. The pump algorithm may also use a negative proportioning control as the measured IV line pressure approaches a threshold value, such as 95% of the IV line pressure safety threshold, and/or the like. Hence, the infusion rate may run at the target infusion rate set point until effectively being limited by IV line safety pressure threshold.

In some non-limiting embodiments or aspects, flow resistance (and/or catheter inner diameter derived from flow resistance) may be determined based on the Poiseuille equation, which is defined according to the following Equation (1):

$$Δ p = \frac{8 μ LQ}{π {R}^{4}} \qquad ( 1 )$$

where Δp is a pressure difference between ends of a flow path or pipe (e.g., between ends of a catheter, etc.), μ is a dynamic viscosity, L is a length of the flow path or pipe (e.g., a length of the catheter, etc.), Q is a volumetric flow rate, π is pi, and r is a radius of the flow path or pipe (e.g., a radius of the catheter, etc.).

In some non-limiting embodiments or aspects, flow resistance (and/or catheter inner diameter derived from flow resistance) for a turbulent flow may be determined based on the Darcy-Weisbach equation, which addresses a correlation between a pressure drop in a fluid flowing through a long cylindrical pipe, a diameter of the pipe, and the velocity of the fluid, and which is defined according to the following Equation (2):

$$\frac{Δ p}{L} = {f}_{D} \times \frac{ρ}{2} \times \frac{{〈 v 〉}^{2}}{D} \qquad ( 1 )$$

where Δp is a pressure difference between ends of a flow path or pipe (e.g., between ends of a catheter, etc.), ρ is a density of the fluid, D is a hydraulic diameter of the flow path or pipe (e.g., a hydraulic diameter of the catheter, etc.), <v> is a mean flow velocity (which may be experimentally measured as a volumetric flow rate Q per unit of cross-sectional wetted area), and f_{D} is the Darcy friction factor.

As shown in FIG. 11, at step 1110, process 1100 includes controlling, based on a flow resistance of a catheter, a plunger rod to move to adjust a flow rate of fluid in the catheter to achieve a desired velocity of the fluid in the catheter. For example, controller 906 may control, based on the flow resistance of catheter 804, syringe pump 802 to move plunger rod 804 axially relative to syringe barrel 102 to adjust the flow rate of the fluid to achieve a desired velocity of the fluid in catheter 802. As an example, syringe pump 802 may move, based on the flow resistance of the catheter, plunger rod 104 axially relative to syringe barrel 102 to adjust the flow rate of the fluid to achieve a desired velocity of the fluid in catheter 804, the flow resistance of catheter 804 being determined based on the measurement of the internal syringe pressure and the flow rate. For example, controller 906 may control motor 902 to move plunger rod 104 axially relative to syringe barrel 102 at the known, constant speed. In such an example, controller 906 may control, based on the flow resistance of catheter 804, motor 902 to move plunger rod 104 axially relative to syringe barrel 102 to adjust the flow rate of the fluid to achieve a desired velocity of the fluid in catheter 804. In such an example, controller 906 may control, based on the flow resistance of catheter 804, motor 902 to drive syringe actuator assembly 129 to move plunger rod 104 axially relative to syringe barrel 102 to adjust the flow rate of the fluid by applying a distally directed force to the proximal end of plunger rod 104. For example, flow rate Q and velocity v may be related according to Q=A⁻ v, where A is the cross-sectional area of the flow (e.g., the flow resistance of the catheter, the diameter of the catheter, etc.), and v is an average velocity of the fluid.

In this way, one or more manual activated flushes of syringe pump 802 can be used to “calibrate” syringe pump 802/controller 906 according to a connected catheter by moving plunger rod 104 forward at one or more known, constant speed and measuring the internal syringe pressure and/or flow rate for each of the known constant speeds and, based on these measurements, a flow resistance of the catheter (and/or an estimated catheter inner diameter) can be derived by syringe pump 802/controller 906 (e.g., assuming that catheter 804 is not partly occluded when syringe pump 802 is connected/deployed, etc.). Syringe pump 802/controller 906 may calculate and/or adjust, based on these measurements and/or derivations, the flow rate to achieve a desired velocity of the fluid in catheter 804. Syringe pump 802/controller 906 may store the measurements, derivations, and/or calculations (e.g., in memory 1006, etc.), which may be independent of a syringe reservoir because force sensor 118 may only measure the force applied on flexible membrane 112 in contact with the fluid in syringe barrel 102 (e.g., without measure or being significantly influenced by a sliding friction force between stopper 110 and the inner surface of syringe barrel 102, etc.). In contrast, if a pressure sensor measures the total force needed to push the plunger including the sliding friction force between a stopper and a syringe barrel, the sliding friction, which varies from syringe reservoir to syringe reservoir, contributes to the sensor measurement.

Although embodiments or aspects have been described in detail for the purpose of illustration and description, it is to be understood that such detail is solely for that purpose and that embodiments or aspects are not limited to the disclosed embodiments or aspects, but, on the contrary, are intended to cover modifications and equivalent arrangements that are within the spirit and scope of the appended claims. For example, it is to be understood that the present disclosure contemplates that, to the extent possible, one or more features of any embodiment or aspect can be combined with one or more features of any other embodiment or aspect. In fact, any of these features can be combined in ways not specifically recited in the claims and/or disclosed in the specification. Although each dependent claim listed below may directly depend on only one claim, the disclosure of possible implementations includes each dependent claim in combination with every other claim in the claim set.

What is claimed is:

1. A syringe pressure sensor assembly, comprising:
a syringe barrel;
a plunger rod including an outer support structure and an inner force translation rod;
a stopper secured to the plunger rod, wherein the stopper is axially movable within the syringe barrel, wherein the stopper includes a flexible membrane including a proximal face and a distal face opposite the proximal face and a sidewall surrounding the flexible membrane, wherein the sidewall of the stopper is in fluid tight engagement with an inner surface of the syringe barrel; and
wherein the outer support structure is secured to the sidewall of the stopper, wherein the inner force translation rod contacts the proximal face of the flexible membrane, wherein the flexible membrane moves proximally relative to the sidewall in response to a proximally directed force applied to the distal face of the flexible membrane, and wherein the inner force translation rod transfers, in response to the proximal movement of the flexible membrane, the proximally directed force to a force sensor.

2. The syringe pressure sensor assembly of claim 1, wherein the force sensor is integrated with the inner force translation rod.

3. The syringe pressure sensor assembly of claim 1, wherein the inner force translation rod extends between a proximal end and a distal end, wherein the distal end of the inner force translation rod contacts the proximal face of the flexible membrane, and wherein the proximal end of the inner force translation rod contacts the force sensor.

4. The syringe pressure sensor assembly of claim 1, wherein the outer support structure includes an inner cavity, wherein the inner force translation rod extends within the inner cavity of the outer support structure.

5. The syringe pressure sensor assembly of claim 1, wherein the force sensor includes at least one of the following: a load cell, a carbon powder-based force sensor, a strain gauge, a piezoelectric sensor, or any combination thereof.

6. The syringe pressure sensor assembly of claim 1, wherein the sidewall of the flexible membrane includes at least one protrusion extending radially inward from the sidewall, and wherein a distal end of the outer support structure includes at least one indentation configured to receive the at least one protrusion, and wherein the stopper is secured to the outer support structure via a complementary mating of the at least one protrusion and the at least one indentation.

7. The syringe pressure sensor assembly of claim 1, wherein a first portion of the flexible membrane has a first thickness between the proximal face and the distal face, wherein the first portion of the flexible membrane is surrounded a second portion of the flexible membrane, wherein the second portion of the flexible membrane has a second thickness between the proximal face and the distal face that is less than the first thickness of the first portion, wherein a third portion of the flexible membrane surrounds the first portion and the second portion, wherein the proximal face of the flexible membrane at the third portion thereof is in contact with a rigid stopper core that inhibits the proximal movement of the flexible membrane, and wherein the inner force translation rode contacts the proximal face of the flexible membrane at the first portion of the flexible membrane.

8. The syringe pressure sensor assembly of claim 1, wherein the flexible membrane has one of a dome shape, a conical shape, and a frusto-conical shape, and wherein the proximal face of the flexible membrane is spaced apart from a distal end of the outer support structure.

9. The syringe pressure sensor assembly of claim 1, further comprising:
a syringe actuator assembly configured to apply a distal force to a proximal end of the plunger rod.

10. The syringe pressure sensor assembly of claim 9, wherein the syringe actuator assembly includes an actuator base and an actuator slider that moves linearly relative to the actuator base, wherein the actuator base includes a track, wherein the actuator slider includes a rail that is received within the track and a plunger contact that extends from the rail and contacts the plunger to apply the distal force to the proximal end of the plunger rod in response to the linear movement of the actuator slider relative to the actuator base.

11. The syringe pressure sensor assembly of claim 10, wherein the plunger contact of the actuator slider includes the force sensor.

12. A system, comprising:
a syringe pressure sensor assembly comprising a syringe barrel, a plunger rod including an outer support structure and an inner force translation rod,
a stopper secured to the plunger rod, wherein the stopper is axially movable within the syringe barrel, wherein the stopper includes a flexible membrane including a proximal face and a distal face opposite the proximal face and a sidewall surrounding the flexible membrane, wherein the sidewall of the stopper is in fluid tight engagement with an inner surface of the syringe barrel, and
wherein the outer support structure is secured to the sidewall of the stopper, wherein the inner force translation rod contacts the proximal face of the flexible membrane, wherein the flexible membrane moves proximally relative to the sidewall in response to a proximally directed force applied to the distal face of the flexible membrane, and wherein the inner force translation rod transfers, in response to the proximal movement of the flexible membrane, the proximally directed force to a force sensor, and
a syringe pump including a motor and at least one processor programmed and/or configured to:
control the motor to move the plunger rod axially relative to the syringe barrel at a known, constant speed;
receive, from the force sensor, a measurement of an internal syringe pressure when the plunger rod is moved axially relative to the syringe barrel at the known, constant speed;
obtain a flow rate of a fluid in a catheter connected to the syringe barrel when the plunger rod is moved axially relative to the syringe barrel at the known, constant speed; and
determine, based on the measurement of the internal syringe pressure and the flow rate, a flow resistance of the catheter.

13. The system of claim 12, wherein the syringe pump further includes an encoder connected to the motor that continuously tracks a rotational movement of the motor, and wherein the flow rate of the fluid in the catheter is determined based on the rotational movement of the motor tracked by the encoder.

14. The system of claim 12, wherein the at least one processor is further programmed and/or configured to:
control, based on the flow resistance of the catheter, the motor to move the plunger rod axially relative to the syringe barrel to adjust the flow rate of the fluid to achieve a desired velocity of the fluid in the catheter.

15. The system of claim 12, further comprising:
a syringe actuator assembly configured to apply a distal force to a proximal end of the plunger rod, wherein the at least one processor is programmed and/or configured to control the motor to drive the syringe actuator assembly to move the plunger rod axially relative to the syringe barrel by applying the distal force to the proximal end of the plunger rod.

16. A method, comprising:
providing a syringe pressure sensor having a syringe barrel, a plunger rod including an outer support structure and an inner force translation rod, a stopper secured to the plunger rod, wherein the stopper is axially movable within the syringe barrel, wherein the stopper includes a flexible membrane including a proximal face and a distal face opposite the proximal face and a sidewall surrounding the flexible membrane, wherein the sidewall of the stopper is in fluid tight engagement with an inner surface of the syringe barrel, and
wherein the outer support structure is secured to the sidewall of the stopper, wherein the inner force translation rod contacts the proximal face of the flexible membrane, wherein the flexible membrane moves proximally relative to the sidewall in response to a proximally directed force applied to the distal face of the flexible membrane, and wherein the inner force translation rod transfers, in response to the proximal movement of the flexible membrane, the proximally directed force to a force sensor;
moving, with a syringe pump, the plunger rod axially relative to the syringe barrel at a known, constant speed;
measuring, with the force sensor, an internal syringe pressure when the plunger rod is moved axially relative to the syringe barrel at the known, constant speed;
measuring, with the syringe pump, a flow rate of a fluid in a catheter connected to the syringe barrel when the plunger rod is moved axially relative to the syringe barrel at the known, constant speed; and
moving, with the syringe pump, based on a flow resistance of the catheter, the plunger rod axially relative to the syringe barrel to adjust the flow rate of the fluid to achieve a desired velocity of the fluid in the catheter, wherein the flow resistance of the catheter is determined based on the measurement of the internal syringe pressure and the flow rate.

17. The method of claim 16, further comprising:
providing a syringe actuator assembly configured to apply a distal force to a proximal end of the plunger rod, wherein syringe pump drives the syringe actuator assembly to move the plunger rod axially relative to the syringe barrel by applying the distal force to the proximal end of the plunger rod.

18. A method, comprising:
controlling, with at least one processor, a syringe pump to move a plunger rod of a syringe axially relative to a syringe barrel of the syringe at a known, constant speed;
receiving, with the at least one processor, from a force sensor, an internal syringe pressure measured when the plunger rod is moved axially relative to the syringe barrel at the known, constant speed;
obtaining, with the at least one processor, from the syringe pump, a flow rate of a fluid in a catheter connected to the syringe barrel determined when the plunger rod is moved axially relative to the syringe barrel at the known, constant speed;
determining, with at least one processor, based on the measurement of the internal syringe pressure and the flow rate, a flow resistance of the catheter; and
controlling, with the at least one processor, based on the flow resistance of the catheter, the syringe pump to move the plunger rod axially relative to the syringe barrel to adjust the flow rate of the fluid to achieve a desired velocity of the fluid in the catheter.

19. The method of claim 18, wherein the flow rate of the fluid in the catheter is determined based on a rotational movement of a motor of the syringe pump.

20. A system, comprising:
at least one processor programmed and/or configured to:
control a syringe pump to move a plunger rod of a syringe axially relative to a syringe barrel of the syringe at a known, constant speed;
receive, from a force sensor, an internal syringe pressure measured when the plunger rod is moved axially relative to the syringe barrel at the known, constant speed;
obtain, from the syringe pump, a flow rate of a fluid in a catheter connected to the syringe barrel determined when the plunger rod is moved axially relative to the syringe barrel at the known, constant speed;
determine, based on the measurement of the internal syringe pressure and the flow rate, a flow resistance of the catheter; and
control, based on the flow resistance of the catheter, the syringe pump to move the plunger rod axially relative to the syringe barrel to adjust the flow rate of the fluid to achieve a desired velocity of the fluid in the catheter.
